(19) United States
(12) Patent Application Publication
(10) Pub. No.: US 2024/0269116 A1
(43) Pub. Date: Aug. 15, 2024

(54) USE OF MMP INHIBITORS FOR TREATING ACUTE RESPIRATORY DISTRESS SYNDROME

(71) Applicant: Foresee Pharmaceuticals Co., Ltd., Taipei City (TW)

(72) Inventors: Yuhua LI, Newark, DE (US); Benjamin CHIEN, Newark, DE (US)

(21) Appl. No.: 18/568,031

(22) PCT Filed: Jun. 7, 2022
(86) PCT No.: PCT/US2022/072789

§ 371 Date: Dec. 7, 2023

Related U.S. Application Data
(60) Provisional application No. 63/208,268, filed on Jun. 8, 2021.

(51) Int. Cl. A61K 31/4178; A61K 47/40; A61P 31/14

(52) U.S. Cl. CPC A61K 31/4178; A61K 47/40; A61P 31/14

(57) ABSTRACT

Methods of treating acute respiratory distress syndrome (ARDS) by administration of a matrix metalloproteinase (MMP) inhibitor are described.

### TECHNICAL FIELD

The application relates to methods of treating acute respiratory distress syndrome (ARDS) by administration of a matrix metalloproteinase (MMP) inhibitor.

### BACKGROUND

Acute respiratory distress syndrome (ARDS) is a life-threatening condition where the lungs cannot provide the body's vital organs with enough oxygen when fluid builds up in the tiny, elastic air sacs in the lungs. It's usually a complication of a serious existing health condition.

This means most people are already in hospital by the time they develop ARDS. Symptoms of ARDS can include: severe shortness of breath, rapid and shallow breathing, tiredness, drowsiness or confusion, and feeling faint.

The mechanical cause of ARDS is fluid leaked from the smallest blood vessels in the lungs into the tiny air sacs where blood is oxygenated. Normally, a protective membrane keeps this fluid in the vessels. Severe illness or injury, however, can cause damage to the membrane, leading to the fluid leakage of ARDS. Common underlying causes of ARDS include sepsis, severe chest injury, inhalation of harmful substances, severe pneumonia, acute pancreatitis, adverse reaction to a blood transfusion, as well as coronavirus disease 2019 (COVID-19).

According to the United States Centers for Disease Control (updated 30 Jun. 2020, https://www.cdc.gov/coronavirus/2019-ncov/hcp/clinical-guidance-management-patients.html), approximately 3% to 17% of COVID-19 patients develop ARDS. Among hospitalized patients, that number increases to 20% to 40%, and up to 67% to 85% for those admitted to the intensive care unit (ICU).

Therefore, under the current pandemic of COVID-19, there is a need to develop a safe and effective means for treating ARDS, especially for severe to critical COVID-19 patients.

### BRIEF SUMMARY

Disclosed herein are methods of treatment of acute respiratory distress syndrome (ARDS) by administration of a matrix metalloproteinase (MMP) inhibitor.

In one general aspect, the application relates to a method of treating acute respiratory distress syndrome (ARDS) in a patient in need thereof, the method comprising administering to the patient a pharmaceutical composition in a therapeutically effective amount, wherein the pharmaceutical composition comprises a cyclodextrin and a compound of formula (F-I) or a pharmaceutically acceptable salt thereof:

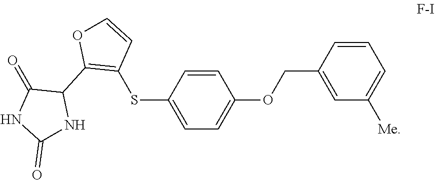 [SMILES: Cc1cccc(COc2ccc(Sc3ccoc3C3NC(=O)NC3=O)cc2)c1]

In some embodiments, a total dosage of the compound of formula (F-I) or the pharmaceutically acceptable salt thereof administered per administration is about 50 mg to 500 mg.

In some embodiments, a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

In some embodiments, the pharmaceutical composition is administered once or twice per day.

In some embodiments, the pharmaceutical composition is administered once per day. In such embodiments, the total dosage of the compound of formula (F-I) or the pharmaceutically acceptable salt thereof administered per day is about 100 mg to about 1000 mg, or any dosage in between.

In some embodiments, the pharmaceutical composition is administered once per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the pharmaceutical composition is administered once per day. In such embodiments, the total dosage of the compound of formula (F-I) or the pharmaceutically acceptable salt thereof administered per day is about 200 mg to about 600 mg, or any dosage in between.

In some embodiments, the pharmaceutical composition is administered twice per day. In such embodiments, the total dosage of the compound of formula (F-I) or the pharmaceutically acceptable salt thereof administered per day is about 100 mg to about 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 100 ng, 200 mg, 300 mg, 400 mg, 500 mg, 600 mg, 700 mg, 800 mg, 900 mg, or 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 200 mg to about 600 mg, or any dosage in between.

In some embodiments, the pharmaceutical composition is administered orally or via a nasogastric tube.

In some embodiments, the pharmaceutical composition comprises the compound of formula (F-I).

In some embodiments, the cyclodextrin is a hydroxypropyl beta-cyclodextrin (HPBCD).

In some embodiments, the pharmaceutical composition has a molar ratio of the compound of formula (F-I) to the cyclodextrin is from 1:0.1 to 1:10.

In some embodiments, the ARDS is caused by coronavirus, preferably caused by SARS-CoV-2.

In some embodiments, the method is a clinically proven safe treatment.

In some embodiments, the method is a clinically proven effective treatment.

In another general aspect, the application relates to a method of treating acute respiratory distress syndrome (ARDS) in a patient in need thereof, the method comprising administering to the patient a compound of formula (F-I) or a pharmaceutically acceptable salt thereof:

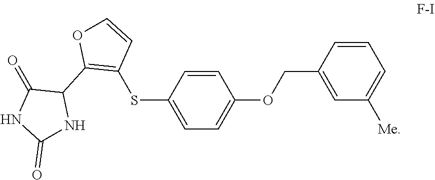 [SMILES: Cc1cccc(COc2ccc(Sc3ccoc3C3NC(=O)NC3=O)cc2)c1]

In some embodiments, a total dosage of the compound of formula (F-I) or the pharmaceutically acceptable salt thereof administered per administration is about 50 mg to 500 mg.

In some embodiments, a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

In some embodiments, the compound of formula (F-I) or the pharmaceutically acceptable salt thereof is administered once or twice per day.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg to about 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 200 mg to about 600 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg to about 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 200 mg to about 600 mg, or any dosage in between.

In some embodiments, the compound of formula (F-I) or the pharmaceutically acceptable salt thereof is administered orally or via a nasogastric tube.

In some embodiments, the ARDS is caused by coronavirus, preferably caused by SARS-CoV-2.

In some embodiments, the method is a clinically proven safe treatment.

In some embodiments, the method is a clinically proven effective treatment.

In another general aspect, the application relates to a method of treating a patient with COVID-19 with associated acute respiratory distress syndrome (ARDS), the method comprising administering to the patient a pharmaceutical composition in a therapeutically effective amount, wherein the pharmaceutical composition comprises a cyclodextrin and a compound of formula (F-I) or a pharmaceutically acceptable salt thereof:

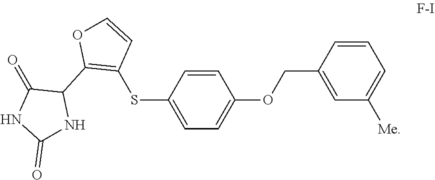 [SMILES: Cc1cccc(COc2ccc(Sc3ccoc3C3NC(=O)NC3=O)cc2)c1]

In some embodiments, a total dosage of the compound of formula (F-I) or the pharmaceutically acceptable salt thereof administered per administration is about 50 mg to 500 mg.

In some embodiments, a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

In some embodiments, the pharmaceutical composition is administered once or twice per day.

In some embodiments, the pharmaceutical composition is administered once per day. In such embodiments, the total dosage of the compound of formula (F-I) or the pharmaceutically acceptable salt thereof administered per day is about 100 mg to about 1000 mg, or any dosage in between.

In some embodiments, the pharmaceutical composition is administered once per day. In such embodiments, the total dosage of the compound of formula (F-I) or the pharmaceutically acceptable salt thereof administered per day is about 100 ng, 200 mg, 300 mg, 400 mg, 500 mg, 600 mg, 700 mg, 800 mg, 900 mg, or 1000 mg, or any dosage in between.

In some embodiments, the pharmaceutical composition is administered once per day. In such embodiments, the total dosage of the compound of formula (F-I) or the pharmaceutically acceptable salt thereof administered per day is about 200 mg to about 600 mg, or any dosage in between.

In some embodiments, the pharmaceutical composition is administered twice per day. In such embodiments, the total dosage of the compound of formula (F-I) or the pharmaceutically acceptable salt thereof administered per day is about 100 mg to about 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 200 mg to about 600 mg, or any dosage in between.

In some embodiments, the pharmaceutical composition is administered orally or via a nasogastric tube.

In some embodiments, the pharmaceutical composition comprises the compound of formula (F-I).

In some embodiments, the cyclodextrin is a hydroxypropyl beta-cyclodextrin (HPBCD).

In some embodiments, the pharmaceutical composition has a molar ratio of the compound of formula (F-I) to the cyclodextrin is from 1:0.1 to 1:10.

In some embodiments, the ARDS is caused by coronavirus, preferably caused by SARS-CoV-2.

In some embodiments, the method is a clinically proven safe treatment.

In some embodiments, the method is a clinically proven effective treatment.

In some embodiments, the method further comprises administering to the patient one or more additional drugs or treatments for treating COVID-19.

In another general aspect, the application relates to a method of treating a patient with COVID-19 with associated acute respiratory distress syndrome (ARDS), the method comprising administering to the patient a compound of formula (F-I) or a pharmaceutically acceptable salt thereof:

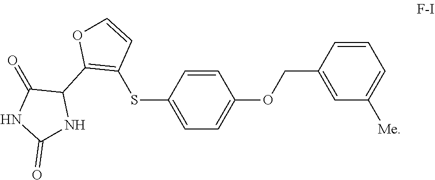 [SMILES: Cc1cccc(COc2ccc(Sc3ccoc3C3NC(=O)NC3=O)cc2)c1]

In some embodiments, a total dosage of the compound of formula (F-I) or the pharmaceutically acceptable salt thereof administered per administration is about 50 mg to 500 mg.

In some embodiments, a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

In some embodiments, the compound of formula (F-I) or the pharmaceutically acceptable salt thereof is administered once or twice per day.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg to about 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 200 mg to about 600 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg to about 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 200 mg to about 600 mg, or any dosage in between.

In some embodiments, the compound of formula (F-I) or the pharmaceutically acceptable salt thereof is administered orally or via a nasogastric tube.

In some embodiments, the ARDS is caused by coronavirus, preferably caused by SARS-CoV-2.

In some embodiments, the method is a clinically proven safe treatment.

In some embodiments, the method is a clinically proven effective treatment.

In some embodiments, the method further comprises administering to the patient one or more additional drugs or treatments for treating COVID-19.

The details of one or more embodiments of the invention are set forth in the description below. Other features and advantages will be apparent from the following detailed description, and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

The patent or application file contains at least one drawing executed in color. Copies of this patent or patent application publication with color drawing(s) will be provided by the Office upon request and payment of the necessary fee.

FIGS. 1A-B show that the compound of formula (F-I) reduced the total inflammatory cell counts (FIG. 1A) and neutrophils (FIG. 1B) in bronchoalveolar lavage (BAL) fluid in acute lung injury (ALI) model. For FIG. 1A: **p<0.01 vs model; ***p<0.001 vs model; ###p<0.001 vs F-I-1 mpk-8 h; $$p<0.001 vs F-I-3 mpk-8 h; &&p<0.05 vs F-I-10 mpk-4 h. For FIG. 1B: *p<0.05 vs model; **p<0.01 vs model; ***p<0.001 vs model; ##p<0.01 vs F-I-1 mpk-8 h; ###p<0.01 vs F-I-1 mpk 8 h; $$p<0.01 vs F-I-3 mpk-8 h; &p<0.05 vs F-I-10 mpk-4 h.

FIGS. 2A-J show changes in lung histology at 24 hours of ALI: sham animal (FIG. 2A); vehicle animal (FIG. 2B); dexamethasone (DEX) at 4 hours of ALI (FIG. 2C); DEX at 8 hours of ALI (FIG. 2D); compound of formula (F-I) 1 mg/kg at 4 hours of ALI (FIG. 2E); compound of formula (F-I) 3 mg/kg at 4 hours of ALI (FIG. 2F); compound of formula (F-I) 10 mg/kg at 4 hours of ALI (FIG. 2G); compound of formula (F-I) 1 mg/kg at 8 hours of ALI (FIG. 2H); compound of formula (F-I) 3 mg/kg at 8 hours of ALI (FIG. 2I); compound of formula (F-I) 10 mg/kg at 8 hours of ALI (FIG. 2J). Blue arrow: normal alveolar wall; black arrow: arteriole with inflammation; red arrow: alveolar wall with inflammatory infiltration. Hematoxylin and eosin staining, magnification of images: ×200.

FIG. 3 shows a reduction in total lung injury with DEX and the compound of formula (F-I) treatment at all different times of ALI and at different dosages. ***p<0.001 vs model.

FIG. 4 shows a reduction in bronchial and arteriole damages with DEX and the compound of formula (F-I) treatment at all different times of ALI and at different dosages. *p<0.05 vs model; **p<0.01 vs model; ***p<0.001 vs model.

FIG. 5 shows a reduction in alveolar damages with DEX and the compound of formula (F-I) treatment at all different times of ALI and at different dosages. *p<0.05 vs model; **p<0.01 vs model; ***p<0.001 vs model.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

### Definitions

It must be noted that as used herein and in the appended claims, the singular forms “a,” “an,” and “the” include plural reference unless the context clearly dictates otherwise.

As used herein, the term “about” preceding a numerical value or a series of numerical values means ±10% of the numerical value unless otherwise indicated. For example, “about 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, unless otherwise noted, the term “clinically proven” (used independently or to modify the term “safe” or “effective”) shall mean that it has been proven by a clinical study in human subjects wherein the clinical study has met the approval standards of U.S. Food and Drug Administration, European Medicines Evaluation Agency (EMEA), or a corresponding national regulatory agency. In one embodiment of the application, the clinical study is a Phase 2/3, randomized, double-blind, placebo-controlled, multicenter study to evaluate the efficacy and safety of the compound of formula (F-I) in patients with severe to critical COVID-19 with associated acute respiratory distress syndrome (ARDS).

The term “clinically proven safe,” as it relates to a dose, dosage regimen, treatment or method with an MMP inhibitor of the present invention (e.g., the compound of formula (F-I)), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator.

As used herein, the phrases “adverse event (AE),” “treatment-emergent adverse event,” “adverse reaction,” and “adverse effect” mean any harm, unfavorable, unintended or undesired sign or outcome associated with or caused by administration of a pharmaceutical composition or therapeutic agent. However, abnormal values or observations are not reported as adverse events unless considered clinically significant by an investigator or a medical doctor.

As used herein, the phrases “serious adverse event (SAE)” and “serious adverse effect” mean any adverse event that is serious, as defined by the Food and Drug Administration (FDA) Code of Federal Regulations (CFR), Chapter 21. A SAE can be any AE or suspected adverse reaction that in the view of an investigator or a medical doctor, results in any of the following outcomes: death, a life threatening adverse event, inpatient hospitalization or prolongation of existing hospitalization, a persistent or significant incapacity or substantial disruption of the ability to conduct normal life functions, or a congenital anomaly/birth defect. Important medical events that may not result in death, be life threatening, or require hospitalization may be considered serious when, based upon appropriate medical judgment, they may jeopardize the patient or subject and may require medical or surgical intervention to prevent one of the outcomes listed in the above definition.

As used herein, when referring to safety assessment of the administration of the MMP inhibitor, “clinically significant changes” means clinically apparent changes as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use. Such changes can be measured by physical examination, such as clinical laboratory assessments, such as clinical chemistry, hematology, and urinalysis; vital signs, such as body temperature, heart rate, respiratory rate, blood pressure and peripheral capillary oxygen saturation (SpO₂); physical examination, such as assessments of general condition, skin, eyes/ears/nose/mouth/throat, neck/thyroid, chest/lungs, heart, vascular system, lymph nodes, abdomen, extremities, nervous systems/reflexes, musculoskeletal system, and spine; and electrocardiogram (ECG) monitoring, including 12 lead safety ECGs.

As used herein, “treatment” or “treat” refers to the treatment of a disease, disorder, or medical condition (such as acute respiratory distress syndrome [ARDS]), in a patient, such as a mammal (particularly a human) which includes one or more of the following:
(a) preventing the disease, disorder, or medical condition from occurring, i.e., preventing the reoccurrence of the disease or medical condition or prophylactic treatment of a patient that is pre-disposed to the disease or medical condition;
(b) ameliorating the disease, disorder, or medical condition, i.e., eliminating or causing regression of the disease, disorder, or medical condition in a patient, including counteracting the effects of other therapeutic agents;
(c) suppressing the disease, disorder, or medical condition, i.e., slowing or arresting the development of the disease, disorder, or medical condition in a patient; or
(d) alleviating the symptoms of the disease, disorder, or medical condition in a patient.

The terms “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, the compound of formula (F-I) can be administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, the compound of formula (F-I) can be administered to achieve an improvement in a subject's condition related to acute respiratory distress syndrome (ARDS). Improvement can be indicated by an improvement in an index of disease activity, by amelioration of clinical symptoms or by any other measure of disease activity. Examples of such index are, but are not limited to, proportion of patients alive, portion of patients on invasive ventilation, portion of patients not requiring non-invasive or invasive ventilation, quantitative assessment of lung fibrosis, number of ventilator-free days, number of intensive care unit (ICU)-free days, number of days in the hospital, change from baseline in peripheral capillary oxygen saturation, change from baseline in PaO₂/FiO₂, proportion of patients with disease progression (defined as a ≥2-point decrease in the NIAID 8-point ordinal scale score or death), proportion of patients who improve by 22 points on the NIAID 8-point ordinal scale scores, pulmonary function, change from baseline in eGFR, blood urea nitrogen, and serum creatinine, and change from baseline in HRQoL as measured by the EQ-5D-5L.

The term “therapeutically effective amount” means an amount sufficient to effect treatment when administered to a patient in need of treatment.

### Methods of Treatment

In one general aspect, the application relates to a method of treating acute respiratory distress syndrome (ARDS) in a patient in need thereof, the method comprising administering to the patient a pharmaceutical composition in a therapeutically effective amount, wherein the pharmaceutical composition comprises a cyclodextrin and a compound of formula (F-I) or a pharmaceutically acceptable salt thereof:

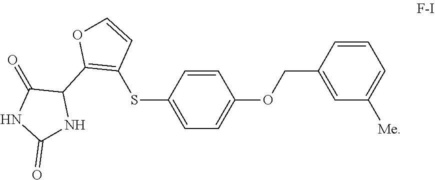 [SMILES: Cc1cccc(COc2ccc(Sc3ccoc3C3NC(=O)NC3=O)cc2)c1]

According to the embodiments of the invention, the compound of formula (F-I) has activity as a matrix metalloproteinase (MMP) inhibitor. The compound of formula (F-I), its synthesis, biologic activities, uses or other related information thereof are described, for example, in U.S. Patent Application Publication No. US 2006/0041000, published on Feb. 23, 2006, the content of which is hereby incorporated by reference in its entirety. In addition, the pharmaceutical compositions comprising the compound of formula (F-I) are described, for example, in International Patent Application Publication No. WO 2018/035459, published on Feb. 22, 2018, the content of which is hereby incorporated by reference in its entirety.

The total dosage of the compound of formula (F-I) or the pharmaceutically acceptable salt thereof per administration is selected so as to provide safe/effective administration and/or safe/effective treatment as determined in clinical trials. According to embodiments of the invention, a total dosage of the compound of formula (F-I) or the pharmaceutically acceptable salt thereof administered is from about 50 mg to about 500 mg per administration, for example, 50 mg, 100 mg, 150 mg, 200 mg, 250 mg, 300 mg, 350 mg, 400 mg, 450 mg, or 500 mg, or any dosage in between.

In preferred embodiments, a total dosage of the compound of formula (F-I) or the pharmaceutically acceptable salt thereof administered per administration is about 100 mg to about 300 mg per administration, for example 100 mg, 150 mg, 200 mg, 250 mg, or 300 mg, or any dosage in between.

In some embodiments, the pharmaceutical composition is administered to the subject in an amount sufficient to provide from about 100 mg per day to about 1000 mg per day, for example, 00 mg, 200 mg, 300 mg, 400 mg, 500 mg, 600 mg, 700 mg, 800 mg, 900 mg, or 1000 mg, or any dosage in between, of the compound of formula (F-I) or a pharmaceutically acceptable salt thereof.

In preferred embodiments, the pharmaceutical composition is administered to the subject in an amount sufficient to provide from about 200 mg per day to about 600 mg per day, for example, 200 mg, 250 mg, 300 mg, 350 mg, 400 mg, 450 mg, 500 mg, 550 mg, or 600 mg, or any dosage in between, of the compound of formula (F-I) or a pharmaceutically acceptable salt thereof.

According to the embodiments of the invention, the pharmaceutical composition can be administered once per day, twice per day, three times per day, once per week, twice per week, etc.

In some embodiments, the pharmaceutical composition is administered once per day. In such embodiments, the total dosage of the compound of formula (F-I) or the pharmaceutically acceptable salt thereof administered per day is about 100 mg to about 1000 mg, for example, 100 ng, 200 mg, 300 mg, 400 mg, 500 mg, 600 mg, 700 mg, 800 mg, 900 mg, or 1000 mg, or any dosage in between.

In some embodiments, the pharmaceutical composition is administered once per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the pharmaceutical composition is administered twice per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the pharmaceutical composition is administered orally or via a nasogastric tube.

In some embodiments, the pharmaceutically acceptable salt of the compound of formula (F-I) means a salt that is acceptable for administration to a patient or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

According to the embodiments of the invention, the ARDS can be caused by illness or injury, such as virus infection, sepsis, inhalation of harmful substances, severe pneumonia, pancreatitis, massive blood transfusions, and trauma to head, chest or other areas of the body.

In preferred embodiments, the ARDS is caused by coronavirus, such as severe acute respiratory syndrome (SARS)-CoV-1, middle east respiratory syndrome (MERS)-CoV, and SARS-CoV-2.

In some embodiments, the patient is diagnosed with COVID-19.

In some embodiments, the method is a clinically proven safe treatment.

In some embodiments, the administration of the pharmaceutical composition does not result in a serious adverse effect. In certain embodiments, the administration of the pharmaceutical composition does not result in an adverse effect.

In some embodiments, the method is a clinically proven effective treatment.

In another general aspect, the application relates to a method of treating acute respiratory distress syndrome (ARDS) in a patient in need thereof, the method comprising administering to the patient a compound of formula (F-I) or a pharmaceutically acceptable salt thereof:

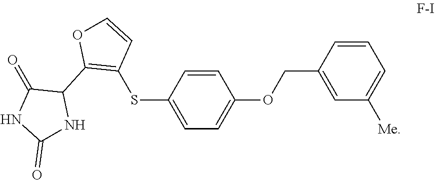 [SMILES: Cc1cccc(COc2ccc(Sc3ccoc3C3NC(=O)NC3=O)cc2)c1]

According to embodiments of the invention, a total dosage of the compound of formula (F-I) or the pharmaceutically acceptable salt thereof administered is from about 50 mg to about 500 mg per administration, for example, 50 mg, 100 mg, 150 mg, 200 mg, 250 mg, 300 mg, 350 mg, 400 mg, 450 mg, or 500 mg, or any dosage in between.

In preferred embodiments, a total dosage of the compound of formula (F-I) or the pharmaceutically acceptable salt thereof administered per administration is about 100 mg to about 300 mg per administration, for example 100 mg, 150 mg, 200 mg, 250 mg, or 300 mg, or any dosage in between.

In some embodiments, the total dosage of the compound of formula (F-I) or the pharmaceutically acceptable salt thereof administered per day is from about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In preferred embodiments, the total dosage of the compound of formula (F-I) or the pharmaceutically acceptable salt thereof administered per day is from about 200 mg to about 600 mg, for example, 200 mg, 250 mg, 300 mg, 350 mg, 400 mg, 450 mg, 500 mg, 550 mg, or 600 mg, or any dosage in between.

According to the embodiments of the invention, the compound of formula (F-I) or the pharmaceutically acceptable salt thereof can be administered once per day, twice per day, three times per day, once per week, twice per week, etc.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the compound of formula (F-I) or the pharmaceutically acceptable salt thereof is administered orally or via a nasogastric tube.

In some embodiments, the pharmaceutically acceptable salt of the compound of formula (F-I) means a salt that is acceptable for administration to a patient or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

According to the embodiments of the invention, the ARDS can be caused by illness or injury, such as virus infection, sepsis, inhalation of harmful substances, severe pneumonia, pancreatitis, massive blood transfusions, and trauma to head, chest or other areas of the body.

In preferred embodiments, the ARDS is caused by coronavirus, such as severe acute respiratory syndrome (SARS)-CoV-1, middle east respiratory syndrome (MERS)-CoV, and SARS-CoV-2.

In some embodiments, the patient is diagnosed with COVID-19.

In some embodiments, the method is a clinically proven safe treatment.

In some embodiments, the administration of the compound of formula (F-I) or the pharmaceutically acceptable salt thereof does not result in a serious adverse effect. In certain embodiments, the administration of the compound of formula (F-I) or the pharmaceutically acceptable salt thereof does not result in an adverse effect.

In some embodiments, the method is a clinically proven effective treatment.

In another general aspect, the application relates to a method of treating a patient with COVID-19 with associated acute respiratory distress syndrome (ARDS), the method comprising administering to the patient a pharmaceutical composition in a therapeutically effective amount, wherein the pharmaceutical composition comprises a cyclodextrin and a compound of formula (F-I) or a pharmaceutically acceptable salt thereof:

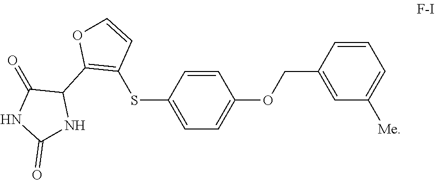 [SMILES: Cc1cccc(COc2ccc(Sc3ccoc3C3NC(=O)NC3=O)cc2)c1]

According to the embodiments of the invention, a total dosage of the compound of formula (F-I) or the pharmaceutically acceptable salt thereof administered is from about 50 mg to about 500 mg per administration, for example, 50 mg, 100 mg, 150 mg, 200 mg, 250 mg, 300 mg, 350 mg, 400 mg, 450 mg, or 500 mg, or any dosage in between.

In preferred embodiments, a total dosage of the compound of formula (F-I) or the pharmaceutically acceptable salt thereof administered per administration is about 100 mg to about 300 mg per administration, for example 100 mg, 150 mg, 200 mg, 250 mg, or 300 mg, or any dosage in between.

In some embodiments, the pharmaceutical composition is administered to the subject in an amount sufficient to provide from about 100 mg per day to about 1000 mg per day, for example, 100 mg, 200 mg, 300 mg, 400 mg, 500 mg, 600 mg, 700 mg, 800 mg, 900 mg, or 1000 mg, or any dosage in between, of the compound of formula (F-I) or a pharmaceutically acceptable salt thereof.

In preferred embodiments, the pharmaceutical composition is administered to the subject in an amount sufficient to provide from about 200 mg per day to about 600 mg per day, for example, 200 mg, 250 mg, 300 mg, 350 mg, 400 mg, 450 mg, 500 mg, 550 mg, or 600 mg, or any dosage in between, of the compound of formula (F-I) or a pharmaceutically acceptable salt thereof.

According to the embodiments of the invention, the pharmaceutical composition can be administered once per day, twice per day, three times per day, once per week, twice per week, etc.

In some embodiments, the pharmaceutical composition is administered once per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the pharmaceutical composition is administered once per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the pharmaceutical composition is administered twice per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the pharmaceutical composition is administered twice per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the pharmaceutical composition is administered orally or via a nasogastric tube.

In some embodiments, the pharmaceutically acceptable salt of the compound of formula (F-I) means a salt that is acceptable for administration to a patient or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

In some embodiments, the method is a clinically proven safe treatment.

In some embodiments, the administration of the pharmaceutical composition does not result in a serious adverse effect. In certain embodiments, the administration of the pharmaceutical composition does not result in an adverse effect.

In some embodiments, the method is a clinically proven effective treatment.

In some embodiments, the patient is diagnosed with severe to critical COVID-19.

In some embodiments, the method further comprises administering to the patient one or more additional drugs or treatments for treating COVID-19. The additional drugs or treatments include standard of care and emergency use authorization (EUA) medications and treatment for COVID-19. Example of the drugs include, but are not limited to, remdesivir (Veklury), dexamethasone, bamlanivimab, casirivimab, imdevimab, and anti-SARS-CoV-2 monoclonal antibodies. Example of the treatments include, but are not limited to, endotracheal intubation, mechanical ventilation, supplemental oxygen delivery, and non-invasive positive pressure ventilation.

In another general aspect, the application relates to a method of treating a patient with COVID-19 with associated acute respiratory distress syndrome (ARDS), the method comprising administering to the patient a compound of formula (F-I) or a pharmaceutically acceptable salt thereof:

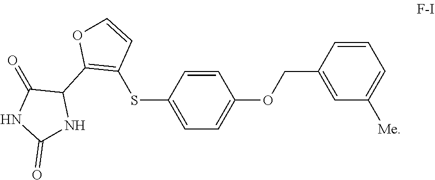 [SMILES: Cc1cccc(COc2ccc(Sc3ccoc3C3NC(=O)NC3=O)cc2)c1]

According to the embodiments of the invention, a total dosage of the compound of formula (F-I) or the pharmaceutically acceptable salt thereof administered is from about 50 mg to about 500 mg per administration, for example, 50 mg, 100 mg, 150 mg, 200 mg, 250 mg, 300 mg, 350 mg, 400 mg, 450 mg, or 500 mg, or any dosage in between.

In preferred embodiments, a total dosage of the compound of formula (F-I) or the pharmaceutically acceptable salt thereof administered per administration is about 100 mg to about 300 mg per administration, for example 100 mg, 150 mg, 200 mg, 250 mg, or 300 mg, or any dosage in between.

In some embodiments, the total dosage of the compound of formula (F-I) or the pharmaceutically acceptable salt thereof administered per day is from about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In preferred embodiments, the total dosage of the compound of formula (F-I) or the pharmaceutically acceptable salt thereof administered per day is from about 200 mg to about 600 mg, for example, 200 mg, 250 mg, 300 mg, 350 mg, 400 mg, 450 mg, 500 mg, 550 mg, or 600 mg, or any dosage in between.

According to the embodiments of the invention, the compound of formula (F-I) or the pharmaceutically acceptable salt thereof can be administered once per day, twice per day, three times per day, once per week, twice per week, etc.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered once per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 100 mg to about 1000 mg, for example, 100 mg, 200 mg, 300 mg, 400 mg, 500 mg, 600 mg, 700 mg, 800 mg, 900 mg, or 1000 mg, or any dosage in between.

In some embodiments, the compound of formula (F-I) or the pharmaceutically acceptable salt thereof is administered twice per day. In such embodiments, the total dosage of the compound of formula (F-I) or the pharmaceutically acceptable salt thereof administered per day is about 200 mg to about 600 mg, for example, 200 mg, 250 mg, 300 mg, 350 mg, 400 mg, 450 mg, 500 mg, 550 mg, or 600 mg, or any dosage in between.

In some embodiments, the compound of formula (F-I) or the pharmaceutically acceptable salt thereof is administered orally or via a nasogastric tube.

In some embodiments, the pharmaceutically acceptable salt of the compound of formula (F-I) means a salt that is acceptable for administration to a patient or a mammal, such as a human (e.g., salts having acceptable mammalian safety for a given dosage regime). Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

In some embodiments, the method is a clinically proven safe treatment.

In some embodiments, the administration of the compound of formula (F-I) or the pharmaceutically acceptable salt thereof does not result in a serious adverse effect. In certain embodiments, the administration of the compound of formula (F-I) or the pharmaceutically acceptable salt thereof does not result in an adverse effect.

In some embodiments, the method is a clinically proven effective treatment.

In some embodiments, the patient is diagnosed with severe to critical COVID-19.

In some embodiments, the method further comprises administering to the patient one or more additional drugs or treatments for treating COVID-19. The additional drugs or treatments include standard of care and emergency use authorization (EUA) medications and treatment for COVID-19. Example of the drugs include, but are not limited to, remdesivir (Veklury), dexamethasone, bamlanivimab, casirivimab, imdevimab, and anti-SARS-CoV-2 monoclonal antibodies. Example of the treatments include, but are not limited to, endotracheal intubation, mechanical ventilation, supplemental oxygen delivery, and non-invasive positive pressure ventilation.

### Pharmaceutical Compositions

According to the embodiments of the invention, the cyclodextrin for use in the pharmaceutical compositions herein is a water soluble unsubstituted or substituted alpha-cyclodextrin (ACD), beta-cyclodextrin (BCD), or gamma-cyclodextrin (GCD). In some embodiments, the beta-cyclodextrin is selected from the group consisting of methyl beta-cyclodextrin (MBCD), hydroxypropyl beta-cyclodextrin (HPBCD), and sulfobutylether beta-cyclodextrin (SBEBCD). In some embodiments, the beta-cyclodextrin is methyl beta-cyclodextrin or hydroxypropyl beta-cyclodextrin. In some embodiments, the gamma-cyclodextrin is hydroxypropyl gamma-cyclodextrin (HPGCD). In one preferred embodiment, the cyclodextrin is hydroxypropyl beta-cyclodextrin (HPBCD) or methyl beta-cyclodextrin (MBCD).

According to the embodiments of the invention, in the pharmaceutical composition, the molar ratio of the compound of formula (F-I) to the cyclodextrin is from 1:0.1 to 1:300, preferably from 1:0.1 to 1:50, and more preferably from 1:0.1 to 1:10.

In some embodiments, the compound of formula (F-I) and hydroxypropyl beta-cyclodextrin (HPBCD) are in the form of an amorphous solid dispersion (ASD).

According to the embodiments of the invention, the pharmaceutical composition of the invention typically contains a therapeutically effective amount of the compound of formula (F-I). Those skilled in the art will recognize, however, that a pharmaceutical composition can contain more than a therapeutically effective amount, e.g., bulk compositions, or less than a therapeutically effective amount, e.g., individual unit doses designed for multiple administration to achieve a therapeutically effective amount.

Typically, such pharmaceutical compositions contains from about 0.1% to about 95% by weight of the compound of formula (F-I); including from about 5% to about 70% by weight of the compound of formula (F-I).

The inventive pharmaceutical compositions comprising the compound of formula (F-I) and a cyclodextrin can further comprise a pharmaceutically acceptable carrier.

As used herein, the term “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. The pharmaceutically acceptable carriers include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Any conventional carrier or excipient may be used in the pharmaceutical compositions of the invention.

The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the carriers or excipients used in the pharmaceutical compositions of this invention are commercially-available. By way of further illustration, conventional formulation techniques are described in Remington: The Science and Practice of Pharmacy, 20th Edition, Lippincott Williams & White, Baltimore, Maryland (2000); and H. C. Ansel et al., Pharmaceutical Dosage Forms and Drug Delivery Systems, 7th Edition, Lippincott Williams & White, Baltimore, Maryland (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

The pharmaceutical compositions of the disclosure are preferably packaged in a unit dosage form. The term “unit dosage form” refers to a physically discrete unit suitable for dosing a patient, i.e., each unit containing a predetermined quantity of active agent calculated to produce the desired therapeutic effect either alone or in combination with one or more additional units. For example, such unit dosage forms may be capsules, tablets, pills, and the like, or unit packages suitable for parenteral administration.

According to the embodiments of the invention, suitable pharmaceutical compositions for oral administration can be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present disclosure as an active ingredient.

When intended for oral administration in a solid dosage form (e.g., as capsules, tablets, pills and the like), the pharmaceutical compositions will typically comprise the active agent (the compound of formula (F-I)), the cyclodextrin, and one or more pharmaceutically-acceptable carriers. Optionally, such solid dosage forms may comprise: fillers or extenders, such as starches, microcrystalline cellulose, lactose, dicalcium phosphate, sucrose, glucose, mannitol, and/or silicic acid; binders, such as carboxymethylcellulose, alginates, gelatin, polyvinyl pyrrolidone, sucrose and/or acacia; humectants, such as glycerol; disintegrating agents, such as crosscarmellose sodium, agar-agar, calcium carbonate, potato or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and/or bentonite clay; lubricants, such as talc, calcium stearate, magnesium stearate, solid polyethylene glycols, sodium lauryl sulfate, and/or mixtures thereof, coloring agents; and buffering agents.

Release agents, wetting agents, coating agents, sweetening, flavoring and perfuming agents, preservatives and antioxidants can also be present in the pharmaceutical compositions of the disclosure. Examples of pharmaceutically-acceptable antioxidants include: water-soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole, butylated hydroxytoluene, lecithin, propyl gallate, alpha-tocopherol, and the like; and metal-chelating agents, such as citric acid, ethylenediamine tetraacetic acid, sorbitol, tartaric acid, phosphoric acid, and the like. Coating agents for tablets, capsules, pills and like, include those used for enteric coatings, such as cellulose acetate phthalate, polyvinyl acetate phthalate, hydroxypropyl methylcellulose phthalate, methacrylic acid, methacrylic acid ester copolymers, cellulose acetate trimellitate, carboxymethyl ethyl cellulose, hydroxypropyl methyl cellulose acetate succinate, and the like.

Pharmaceutical compositions of the invention can also be formulated to provide slow or controlled release of the active agent using, by way of example, hydroxypropyl methylcellulose in varying proportions; or other polymer matrices, liposomes and/or microspheres. In addition, the pharmaceutical compositions of the invention can optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active agent can also be in micro-encapsulated form, if appropriate, with one or more of the above-described excipients.

Suitable liquid dosage forms for oral administration include, by way of illustration, pharmaceutically-acceptable emulsions, microemulsions, solutions, suspensions, syrups and elixirs. Liquid dosage forms typically comprise the active agent and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), oleic acid, glycerol, tetrahydrofuryl alcohol, polyethylene glycols and fatty acid esters of sorbitan, and mixtures thereof. Alternatively, certain liquid formulations can be converted, for example, by spray drying, to a powder, which is used to prepare solid dosage forms by conventional procedures.

Suspensions, in addition to the active ingredient, can contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

According to embodiments of the invention, the pharmaceutical composition can be administered by via a nasogastric tube. In such embodiments, the solid dosage form is opened up (for capsules and pills) or grounded into powders (for tablets), and then delivered as a suspension of solution in liquid, and the liquid dosage is delivered directly or diluted with another liquid.

### EXAMPLES

### Example 1: Effect of Orally Administered Compound of Formula (F-I) on Lipopolysaccharide (LPS)-Induced Mice in the Acute Lung Injury (ALI) Model

The compound of formula (F-I) was evaluated for the therapeutic efficacy in the LPS-induced mice ALI model. In this study, animals were anesthetized with 2.5% isoflurane inhalation. LPS was used to create ALI at the dose of 3 mg/kg in 50 μl via a direct intratracheal injection. For sham animals, an equal volume of saline solution was directly injected into the trachea. After LPS injection, the test article compound of formula (F-I) was dosed at different single oral doses and different therapeutic time points. Male C57BL/6 mice (n=100) were used in this study. DEX was used as positive control. Animals were randomly divided into 10 groups and dose regimens are described in Table 1. At 24 hours after injection, all animals were euthanized and processed for bronchoalveolar lavage (BAL) fluid collection from bilateral lungs and formalin-fixation of lung tissue pathology studies.

TABLE 1
Group Assignment and Dosing Regimen for the ALI Model

| | | | | Concentration | Dosage | Dosage | |
|---|---|---|---|---|---|---|---|
| Group | N | Control | Compound | (mg/mL) | (mL/kg) | (mg/kg) | Regimen |
| 1 | 10 | Sham | Saline | N/A | 10 | N/A | N/A |
| 2 | 10 | LPS | Vehicle | N/A | 10 | N/A | At the same time as LPS |
| 3 | 10 | LPS | DEX | 0.3 | 10 | 3 | At 4 hours of LPS |
| 4 | 10 | LPS | DEX | 0.3 | 10 | 3 | At 8 hours after LPS |
| 5 | 10 | LPS | (I) | 0.1 | 10 | 1 | At 4 hours after LPS |
| 6 | 10 | LPS | (I) | 0.3 | 10 | 3 | At 4 hours after LPS |
| 7 | 10 | LPS | (I) | 1 | 10 | 10 | At 4 hours after LPS |
| 8 | 10 | LPS | (I) | 0.1 | 10 | 1 | At 8 hours after LPS |
| 9 | 10 | LPS | (I) | 0.3 | 10 | 3 | At 8 hours after LPS |
| 10 | 10 | LPS | (I) | 1 | 10 | 10 | At 8 hours after LPS |

ALI = Acute lung injury; DEX = Dexamethasone; LPS = Lipopolysaccharide; N/A = Not applicable.

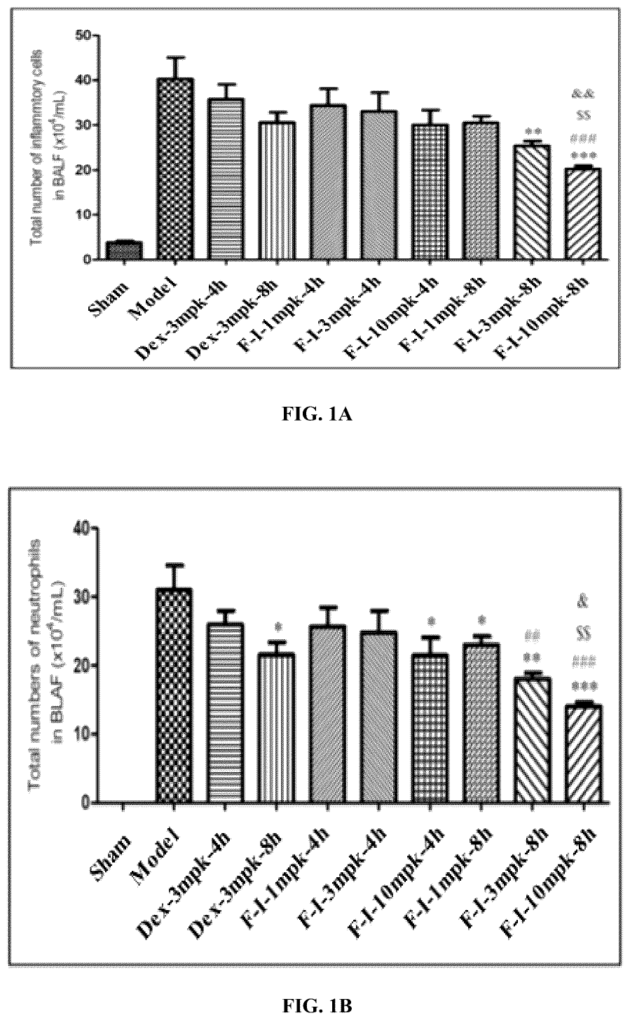

Data showed a significant LPS-induced increase in total inflammatory cells in BAL fluid (FIGS. 1A-B). Most of inflammatory cells were neutrophils. DEX treatment at 4 and 8 hours of ALI had a significant reduction in total inflammatory cells (FIG. 1A) and neutrophils (FIG. 1B) in BAL fluid as compared to Vehicle group. Treatment of DEX at 8 hours of ALI was better than at 4 hours of ALI. Treatment of the compound of formula (F-I) at 4 and 8 hours of ALI had a clear trend of dose-dependent reduction in total inflammatory cells and neutrophils in BAL fluid as compared to Vehicle group in all 3 treatment groups. Treatment of the compound of formula (F-I) at 8 hours of ALI had a better effect than that at 4 hours of ALI. Treatment of the compound of formula (F-I) at a dose of 10 mg/kg showed the best efficacy in BAL fluid analysis.

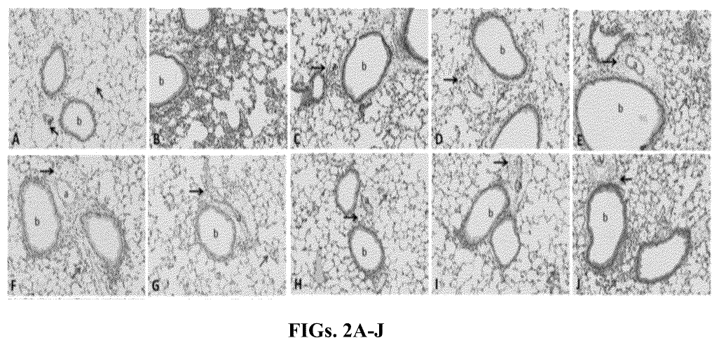

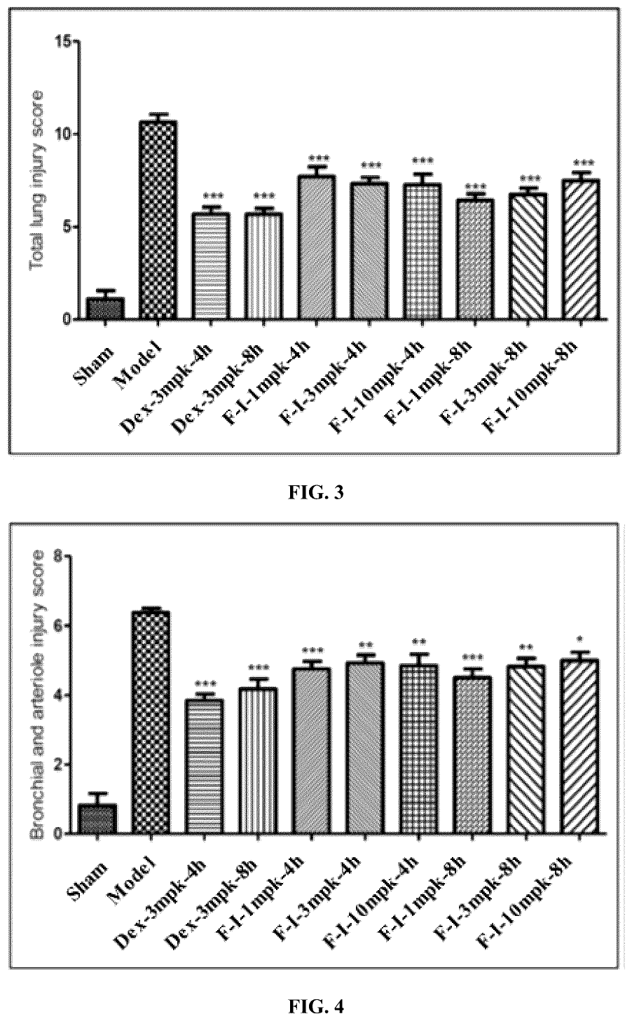

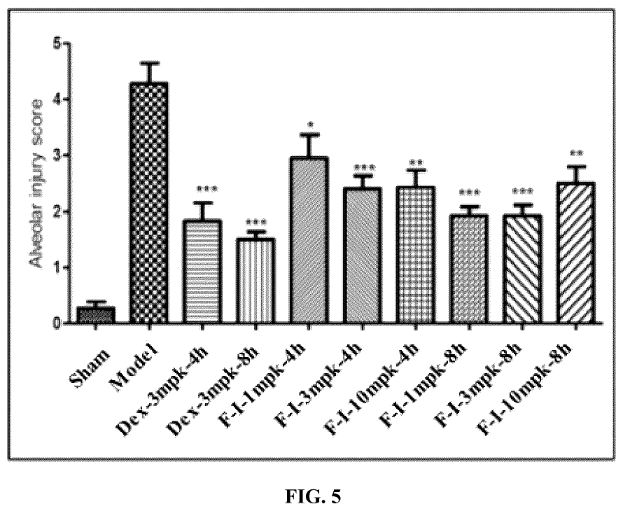

Lung histology evaluation (FIG. 2) at 24 hours of ALI showed a reduction in total lung injury (FIG. 3) that included bronchial, arteriole (FIG. 4) and alveolar (FIG. 5) damages with DEX and compound of formula (F-I) treatment at all different times of ALI and at different dosages. DEX treatment at 8 hours of ALI showed a much better efficacy as compared to that at 4 hours of ALI. Treatment of the compound of formula (F-I) at 8 hours of ALI showed a better efficacy as compared to that at 4 hours of ALI. There was no clear dose-dependent lung tissue protection of either dosing at 4 or 8 hours of ALI.

In conclusion, treatment of the compound of formula (F-I) at 4 or 8 hours of ALI at lower doses (1 mg/kg) via oral gavage still showed a significant therapeutic efficacy, dosing at 8 hours of ALI was slightly better than that at 4 hours of ALI. The efficacy of the compound of formula (F-I) at 10 mg/kg was better at 4 hours and 8 hours of ALI.

### Example 2: A Clinical Study to Evaluate the Efficacy and Safety of the Compound of Formula (F-I) in Patients with Severe to Critical COVID-19 with Associated Acute Respiratory Distress Syndrome (ARDS)

This clinical study is a Phase 2/3, randomized, double-blind, placebo-controlled, multicenter study. The purpose of this study is to evaluate the efficacy and safety of the compound of formula (F-I) in patients with severe to critical COVID-19 with associated acute respiratory distress syndrome (ARDS).

### Objectives

### Primary Objectives:

1) To evaluate the efficacy of the compound of formula (F-I), compared to placebo, in adult patients with severe to critical COVID-19 with associated ARDS, when used with standard of care treatment for COVID-19; and
2) To evaluate the safety and tolerability of the compound of formula (F-I) in adult patients with severe to critical COVID-19 with associated ARDS.

### Secondary Objectives:

1) To evaluate the efficacy of the compound of formula (F-I), compared to placebo, in improving acute COVID-19 outcomes as measured by the change over time in the National Institute of Allergy and Infectious Diseases (NIAID) 8-point ordinal scale for COVID-19 and hospitalization outcomes, when used with standard of care treatment for COVID-19;
2) To evaluate the efficacy of the compound of formula (F-I), compared to placebo, in reducing or preventing pulmonary fibrosis as determined by quantitative high-resolution, non-contrast computed tomography (CT) scan, when used with standard of care treatment for COVID-19;
3) To evaluate the effect of the compound of formula (F-I), compared to placebo, on pulmonary function testing, when used with standard of care treatment for COVID-19;
4) To evaluate the long-term effects of the compound of formula (F-I), compared to placebo, on health-related quality of life (HRQoL) after COVID-19, using the EuroQoL Five Dimension Questionnaire (EQ-5D-5L), when used with standard of care treatment for COVID-19;
5) To evaluate the effect of the compound of formula (F-I), compared to placebo, on renal function in adult patients with severe to critical COVID-19 with associated ARDS; and
6) To assess the pharmacokinetics (PK) of the compound of formula (F-I) in adult patients with severe to critical COVID-19 with associated ARDS.

### Exploratory Objective:

1) To evaluate the effect of the compound of formula (F-I), compared to placebo, on inflammatory and fibrotic biomarkers in blood when used with standard of care treatment for COVID-19.

### Population

The population for this study includes male or female patients ≥18 years with a diagnosis of severe to critical COVID-19 with associated ARDS.

### Study Design and Duration

This is a Phase 2/3, randomized, double-blind, placebo-controlled, multicenter study to evaluate the efficacy and safety of the compound of formula (F-I) in adult patients with severe to critical COVID-19 with associated ARDS. The patients in each phase (Phase 2 and Phase 3) are analyzed separately.

Each phase consists of a Screening Visit, Treatment Period, and Follow-Up Period for a total study duration of approximately 60 days.

### Phase 2

After eligibility is confirmed, approximately 99 patients are randomized in a 1:1:1 ratio to receive the compound of formula (F-I) 100 mg twice daily (BID) or 300 mg BID, or placebo BID for 28 days. During Phase 2, randomized patients are stratified by the use of invasive mechanical ventilation at the time of randomization (yes or no). At least one-third of patients should be on invasive mechanical ventilation to ensure that treatment benefits can be assessed for patients at different severity levels.

Standard of care procedures for mechanical ventilation (e.g., low tidal volume protective mechanical ventilation) and standard of care procedures for weaning from mechanical ventilation will be followed.

Study drug administration begins on Day 1 (Visit 2) following randomization. All patients receive standard of care and/or EUA treatment for COVID-19 in addition to the study drug. Patients continue study drug treatment BID through Day 28. If a patient is discharged from the hospital prior to Day 28, he/she will continue treatment as an outpatient at home with his/her assigned treatment (the compound of formula (F-I) 100 mg BID or 300 mg BID, or placebo BID) until Day 28. Dosing instructions are provided prior to discharge. Although treatment is identical for inpatients and outpatients, patients discharged from the hospital follow a different Schedule of Procedures, characterized by telemedicine visits. The End of Treatment (EOT) Visit on Day 28 is identical for all patients (with the exception of the arterial oxygen partial pressure [PaO2]/fractional inspired oxygen [FiO2] ratio, performed only in patients on invasive or non-invasive ventilation) and includes a high-resolution, non-contrast CT scan, in addition to other assessments.

After treatment is completed, all patients undergo 2 follow-up assessments during the Follow-Up Period. The first follow-up is a telephone visit on Day 45 to assess concomitant medications, adverse events (AEs), and the NIAID 8-point ordinal scale for COVID-19 and hospitalization outcomes score. The second follow-up is an in-person visit on Day 60 and includes a high-resolution, non-contrast CT scan and pulmonary function tests, in addition to other assessments.

### Phase 3

Based on the results of the primary analysis of Phase 2, 1 optimal dose is selected to carry into Phase 3. Once the optimal dose has been selected, the study may proceed to Phase 3. Patients is randomized in a 1:1 ratio to receive the compound of formula (F-I) or placebo for 28 days. Similar procedures are followed as for Phase 2.

### Dosage Forms and Route of Administration

The compound of formula (F-I) is supplied as an oral capsule (amorphous solid dispersion-in-capsule) at a dose strength of 50 mg per capsule. The capsule consists of 25% of the compound of formula (F-I) and 75% of hydroxypropyl beta-cyclodextrin (HPBCD) by weight. The capsules are administered orally or via a nasogastric tube (by opening up the capsules and delivering as a suspension in orange juice).

During Phase 2, patients are randomized in a 1:1:1 ratio to receive the compound of formula (F-I) 100 mg BID or 300 mg BID, or placebo BID for 28 days, starting on Day 1 (Visit 2). Patients in each dose group will receive a total of 6 capsules during each dosing session as follows:
Compound of formula (F-I) 100 mg BID: Two 50 mg capsules and 4 placebo capsules;
Compound of formula (F-I) 300 mg BID: Six 50 mg capsules; and
Placebo: 6 placebo capsules.

Based on the results of the primary analysis of Phase 2, one optimal dose is selected to carry into Phase 3, and patients is randomized in a 1:1 ratio to receive the compound of formula (F-I) or placebo for 28 days. No dose adjustments is performed for renal impairment.

### Efficacy Endpoints

For all endpoints defined by events, the time points are defined as the number of days after randomization.

The primary efficacy endpoint is the proportion of patients alive and not requiring non-invasive or invasive ventilation (i.e., not receiving high-flow nasal cannula, non-invasive positive pressure ventilation, invasive mechanical ventilation, or ECMO) at Day 28, with each dose (Phase 2) or the optimal dose (Phase 3) of the compound of formula (F-I) compared to placebo.

The key secondary efficacy endpoints include the following, with each dose (Phase 2) or the optimal dose (Phase 3) of the compound of formula (F-I) compared to placebo:
Proportion of patients on invasive mechanical ventilation at Day 28;
Proportion of patients alive at Day 28;
Proportion of patients alive and not requiring non-invasive or invasive ventilation (i.e., not receiving high-flow nasal cannula, non-invasive positive pressure ventilation, invasive mechanical ventilation, or ECMO) at Day 60;
Proportion of patients on invasive mechanical ventilation at Day 60; and
Proportion of patients alive at Day 60.

The other secondary efficacy endpoints include the following, with each dose (Phase 2) or the optimal dose (Phase 3) of the compound of formula (F-I) compared to placebo:
Quantitative assessment of lung fibrosis at Day 28 and at Day 60 using high-resolution, non-contrast CT scan;
Proportion of patients who were randomized on invasive mechanical ventilation who are free of invasive mechanical ventilation at Day 28 and at Day 60;
Number of ventilator-free days (i.e., days free of invasive mechanical ventilation) at Day 28 and at Day 60;
Number of intensive care unit (ICU)-free days at Day 28 and at Day 60;
Number of days in the hospital at Day 28 and at Day 60;
Change from baseline in peripheral capillary oxygen saturation at Day 28 and at Day 60;
Change from baseline in PaO2/FiO2 ratio at Day 28 and at Day 60 (patients on invasive or non-invasive ventilation only);
Proportion of patients alive and not hospitalized at Day 28 and at Day 60;
Proportion of patients with disease progression (defined as a ≥2-point decrease in the NIAID 8-point ordinal scale score or death) at Day 28 and at Day 60;
Proportion of patients who improve by ≥2 points on the NIAID 8-point ordinal scale scores at Day 28 and proportion of patients who improve by ≥2 points at Day 60;
Pulmonary function at Day 28 (if possible based on the patient's clinical condition per the judgment of the Investigator) and at Day 60 (all patients) based on the following scores:
   Forced vital capacity (FVC);
   Forced expiratory volume in 1 second (FEV1);
   FEV1/FVC ratios; and
   Diffusing capacity of the lungs for carbon monoxide;
Change from baseline in eGFR, blood urea nitrogen, and serum creatinine at Day 28 and Day 60; and
Change from baseline in HRQoL as measured by the EQ-5D-5L at Day 28 and Day 60.

Exploratory endpoints includes the change from baseline (pre-dose on Day 1) in biomarkers related to pulmonary inflammation, fibrosis and/or lung injury at Day 7, Day 14, Day 28, and Day 60, including but not limited to the following, with each dose (Phase 2) or the optimal dose (Phase 3) of the compound of formula (F-I) compared to placebo:
MMP-12, MMP-7, and MMP-9;
Transforming growth factor-β;
Connective tissue growth factor;
C-reactive protein (CRP);
Cytokines (interferon-γ, tumor necrosis factor-α, interleukin [IL]-6, IL-1β, and C—C motif chemokine ligand 2 [monocyte chemoattractant protein-1]);
Ferritin;
Released N-terminal pro-peptide of type III collagen;
Internal epitope in the 7S domain of type IV collagen;
C-terminal of released C5 domain of type VI collagen 3 chain (endotrophin);
Neo-epitope of MMP-2, 9, and 13-mediated degradation of type I collagen;
Neo-epitope of MMP-2, 9, and 12-mediated degradation of type IV collagen;
Neo-epitope of MMP-2-mediated degradation of type VI collagen;
Neo-epitope of MMP-1 and 8-mediated degradation of CRP; and
Neo-epitope of plasmin-mediated degradation of cross-linked fibrin.

Additional biomarkers not listed here may be evaluated at a future date.

### Pharmacokinetic Endpoint

The PK endpoint is the plasma exposure of the compound of formula (F-I) at each dose level. Sampling times will be as follows:
Day 1 (Visit 2): pre-dose, 1 hour (10 minutes), 3 hours (10 minutes), and 8 hours (±10 minutes) post-morning dose;
Day 4, Day 7, Day 14, and Day 21: within 1 hour (10 minutes) prior to the morning dose; and
Day 28: within 1 hour (10 minutes) prior to the morning dose and 1 hour (10 minutes) post-morning dose.

### Efficacy Assessments

### Arterial Oxygen Partial Pressure/Fractional Inspired Oxygen Ratio

A PaO2, obtained via arterial blood gas is used to calculate the PaO2/FiO2 ratio, only in patients on invasive or non-invasive ventilation.

### Pulmonary Function Tests

Pulmonary function tests (PFTs) is performed at the designed times. PFTs include measurement of FVC, FEV1, FEV1/FVC ratio, and diffusing capacity of the lungs for carbon monoxide.

### Computed Tomography Scan

A high-resolution, non-contrast CT scan is performed at the designed times to assess for lung fibrosis. The CT scan is acquired with a single acquisition of reduced dose (i.e., 6 mSv total). A chest radiograph can be substituted for the high-resolution, non-contrast CT scan at the Screening Visit only for study eligibility.

### Inflammatory and Fibrotic Biomarker Analysis

Biomarker analyses are performed at the designed times. Biomarkers include, but are not limited to, assessments of MMP-12, MMP-7, MMP-9, TGF-β, connective tissue growth factor, CRP, cytokines (IFN-γ, TNF-α, IL-6, IL-1β, and C—C motif chemokine ligand 2 [monocyte chemoattractant protein-1]), ferritin levels, released N-terminal pro-peptide of type III collagen, internal epitope in the 7S domain of type IV collagen, C-terminal of released C5 domain of type VI collagen 3 chain (endotrophin), neo-epitope of MMP-2, 9, and 13-mediated degradation of type I collagen, neo-epitope of MMP-2, 9, and 12-mediated degradation of type IV collagen, neo-epitope of MMP-2-mediated degradation of type VI collagen, neo-epitope of MMP-1 and 8-mediated degradation of CRP, and neo-epitope of plasmin-mediated degradation of cross-linked fibrin in blood.

Blood is collected from patients that have consented to the procedure. The blood samples are collected per the laboratory manual instructions and analyzed. Samples can be stored at the central laboratory for future analyses.

### National Institute of Allergy and Infectious Diseases 8-Point Ordinal Scale

The NIAID 8-point ordinal scale score is assessed at the designed times.

### EuroQoL Five Dimension Questionnaire

HRQoL is assessed using the EQ-5D-5L at the designed times. The baseline EQ-5D-5L should be collected as soon as feasible upon patient enrollment based on historical values prior to onset of COVID-19 symptoms.

### Other Assessments

### Severe Acute Respiratory Syndrome Coronavirus-2 Testing

SARS-CoV-2 testing can be performed at the Screening Visit. The patient must have a documented, laboratory-confirmed SARS-CoV-2 infection as determined by reverse transcriptase polymerase chain reaction (or an equivalent test) immediately prior to or during the current hospitalization to be included in the study.

### Safety Endpoints

The safety endpoints include the following:
Adverse events (AEs) and serious AEs (SAEs);
Clinical laboratory assessments (including clinical chemistry, hematology, and urinalysis);
Vital signs;
Physical examinations; and
12-lead electrocardiograms (ECGs).

AEs are monitored and recorded from the time of the first dose of study drug (Day 1 [Visit 2]) until the end of the study (Day 60). All events occurring prior to the first dose of study drug are recorded as medical history.

Any SAE that is ongoing at the end of the study (Day 60) is followed by the Investigator until the SAE has subsided or until the condition(s) becomes chronic in nature, until the patient begins alternative treatment for COVID-19, until the condition(s) stabilizes (in the case of persistent impairment), or until the patient dies.

As events of progression of the patient's underlying COVID-19 diagnosis are captured as efficacy endpoints, these events should not be reported as AEs or SAEs unless considered related to the study drug or a study procedure or if the outcome is fatal during the study and within the safety reporting period. Additionally, expected events for ARDS that are perceived by the Investigator to occur with reasonable frequency in the day-to-day care of patients with ARDS treated in an ICU setting with mechanical ventilation are not reported as AEs. Examples of events that are expected in the course of ARDS include transient hypoxemia, agitation, delirium, etc. Such events, which are often the focus of prevention efforts as part of standard ICU care, are not considered reportable AEs unless the event is considered by the Investigator to be associated with the study drug or study procedures, is unexpectedly severe or frequent, or is a change of severity for an individual patient with ARDS.

### Safety Assessments

An AE is defined as any untoward medical occurrence in a clinical investigation patient administered a pharmaceutical product, which does not necessarily have a causal relationship with this treatment. An AE can therefore be any unfavorable and/or unintended sign (including an abnormal laboratory finding), symptom, or disease temporally associated with the use of an investigational medicinal product, whether or not related to the investigational medicinal product. All AEs, including observed or volunteered problems, complaints, or symptoms, are to be recorded on the appropriate eCRF.

An AE or adverse reaction is considered serious if, in the view of either the Investigator or Sponsor, it results in any of the following outcomes:
Death;
A life-threatening AE;
Requires hospitalization or prolongation of existing hospitalizations;
A persistent or significant disability/incapacity or substantial disruption of the ability to conduct normal life functions;
A congenital anomaly/birth defect; or
An important medical event.

### Statistical Analysis

Patients who discontinue the study drug remain in the study to have safety and efficacy assessments performed. The data observed on-study are used for analysis regardless of any intercurrent events. Patients who die or discontinue treatment prior to the assessment time point (Day 28 or Day 60) because of lack of efficacy, and cannot have clinical status determined at the time point, are considered treatment failures.

As to the primary efficacy endpoint, the treatment group difference of the response proportions, along with the corresponding 95% confidence interval and p-value, is provided using the logistic regression model (by Guo Y, Wu V, Li X, et al. PharmaSUG China 1st Conference. 2012; Ge M, Durham L K, Meyer R D, et al. Ther. Innov. Regul. Sci. 2011; 45(4):481-493), adjusted for stratification factor (use of invasive mechanical ventilation at the time of randomization [yes or no]). Additional prognostic baseline covariates (e.g., age, region, body mass index) may be added as covariates/factors into the model to increase precision in the estimation of difference in proportions.

The key secondary endpoints are analyzed using the same method as for the primary analysis.

Tipping point method will be used as a sensitivity analysis for evaluating the robustness of the results for the primary endpoint and key secondary endpoints.

PK data are summarized.

Safety data are summarized by actual treatment received, and in total for selected analyses/summaries, based on the Safety Population, and include the following:
AEs and SAEs;
Clinical laboratory assessments (including clinical chemistry, hematology, and urinalysis);
Vital signs;
Physical examinations; and
12-lead ECGs.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present inventions as defined by the specific description.

1. A method of treating acute respiratory distress syndrome (ARDS) in a patient in need thereof, the method comprising administering to the patient a pharmaceutical composition in a therapeutically effective amount, wherein the pharmaceutical composition comprises a cyclodextrin and a compound of formula (F-I) or a pharmaceutically acceptable salt thereof:

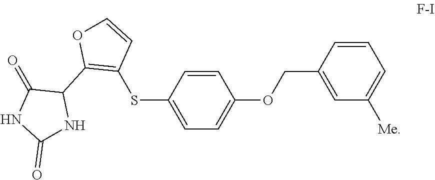 [SMILES: Cc1cccc(COc2ccc(Sc3ccoc3C3NC(=O)NC3=O)cc2)c1]

2. The method of claim 1, wherein a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

3. The method of claim 1 or 2, wherein the pharmaceutical composition is administered once or twice per day.

4. The method of any one of claims 1-3, wherein a total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

5. The method of any one of claims 1-4, wherein the total dosage of the compound of formula (F-I) or the pharmaceutically acceptable salt thereof administered per administration is about 100 to about 300 mg.

6. The method of any one of claims 1-5, wherein the total dosage of the compound of formula (F-I) or the pharmaceutically acceptable salt thereof administered per day is about 200 to about 600 mg.

7. The method of any one of claims 1-6, wherein the pharmaceutical composition is administered orally or via a nasogastric tube.

8. The method of any one of claims 1-7, wherein the pharmaceutical composition comprises the compound of formula (F-I).

9. The method of any one of claims 1-8, wherein the cyclodextrin is a hydroxypropyl beta-cyclodextrin (HPBCD).

10. The method of any one of claims 1-9, wherein a molar ratio of the compound of formula (F-I) to the cyclodextrin is from 1:0.1 to 1:10.

11. The method of any one of claims 1-10, wherein the ARDS is caused by coronavirus, preferably caused by SARS-CoV-2.

12. A method of treating a patient with COVID-19 with associated acute respiratory distress syndrome (ARDS), the method comprising administering to the patient a pharmaceutical composition in a therapeutically effective amount, wherein the pharmaceutical composition comprises a cyclodextrin and a compound of formula (F-I) or a pharmaceutically acceptable salt thereof:

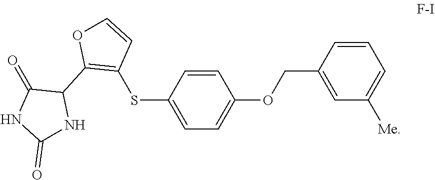 [SMILES: Cc1cccc(COc2ccc(Sc3ccoc3C3NC(=O)NC3=O)cc2)c1]

13. The method of claim 12, wherein a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

14. The method of claim 12 or 13, wherein the pharmaceutical composition is administered once or twice per day.

15. The method of any one of claims 12-14, wherein a total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

16. The method of any one of claims 12-15, wherein the total dosage of the compound of formula (F-I) or the pharmaceutically acceptable salt thereof administered per administration is about 100 to about 300 mg.

17. The method of any one of claims 12-16, wherein the total dosage of the compound of formula (F-I) or the pharmaceutically acceptable salt thereof administered per day is about 200 to about 600 mg.

18. The method of any one of claims 12-17, wherein the pharmaceutical composition is administered orally or via a nasogastric tube.

19. The method of any one of claims 12-18, wherein the pharmaceutical composition comprises the compound of formula (F-I).

20. The method of any one of claims 1-8, wherein the cyclodextrin is a hydroxypropyl beta-cyclodextrin (HPBCD).

21. The method of any one of claims 12-20, wherein a molar ratio of the compound of formula (F-I) to the cyclodextrin is from 1:0.1 to 1:10.

22. The method of any one of claims 12-21, further comprising administering to the patient one or more additional drugs or treatments for treating COVID-19.

23. A method of treating acute respiratory distress syndrome (ARDS) in a patient in need thereof, the method comprising administering to the patient a compound of formula (F-I) or a pharmaceutically acceptable salt thereof:

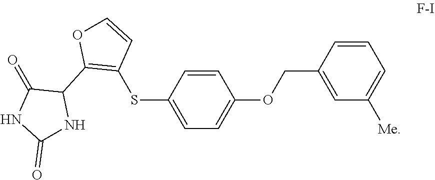 [SMILES: Cc1cccc(COc2ccc(Sc3ccoc3C3NC(=O)NC3=O)cc2)c1]

24. The method of claim 23, wherein a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

25. The method of claim 23 or 24, wherein the compound of formula (F-I) or the pharmaceutically acceptable salt thereof is administered once or twice per day.

26. The method of any one of claims 23-25, wherein a total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

27. The method of any one of claims 23-26, wherein the total dosage of the compound of formula (F-I) or the pharmaceutically acceptable salt thereof administered per administration is about 100 to about 300 mg.

28. The method of any one of claims 23-27, wherein the total dosage of the compound of formula (F-I) or the pharmaceutically acceptable salt thereof administered per day is about 200 to about 600 mg.

29. The method of any one of claims 23-28, wherein the compound of formula (F-I) or the pharmaceutically acceptable salt thereof is administered orally or via a nasogastric tube.

30. The method of any one of claims 23-29, wherein the compound of formula (F-I) is administered to the patient.

31. The method of any one of claims 23-30, wherein the ARDS is caused by coronavirus, preferably caused by SARS-CoV-2.

32. A method of treating a patient with COVID-19 with associated acute respiratory distress syndrome (ARDS), the method comprising administering to the patient a compound of formula (F-I) or a pharmaceutically acceptable salt thereof:

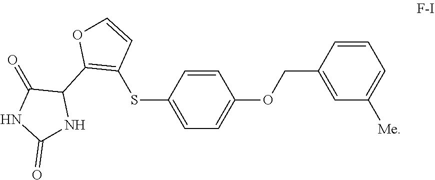 [SMILES: Cc1cccc(COc2ccc(Sc3ccoc3C3NC(=O)NC3=O)cc2)c1]

33. The method of claim 32, wherein a total dosage of the compound of formula (F-I) or the pharmaceutically acceptable salt thereof administered per administration is about 50 mg, 100 mg, 150 mg, 200 mg, 300 mg, 400 mg, or 500 mg, or any dosage in between.

34. The method of claim 32 or 33, wherein the compound of formula (F-I) or the pharmaceutically acceptable salt thereof is administered once or twice per day.

35. The method of any one of claims 32-34, wherein a total dosage of the compound of formula (F-I) or the pharmaceutically acceptable salt thereof administered per day is about 100 mg, 200 mg, 300 mg, 400 mg, 500 mg, 600 mg, 700 mg, 800 mg, 900 mg, or 1000 mg, or any dosage in between.

36. The method of any one of claims 32-35, wherein the total dosage of the compound of formula (F-I) or the pharmaceutically acceptable salt thereof administered per administration is about 100 to about 300 mg.

37. The method of any one of claims 32-36, wherein the total dosage of the compound of formula (F-I) or the pharmaceutically acceptable salt thereof administered per day is about 200 to about 600 mg.

38. The method of any one of claims 32-37, wherein the compound of formula (F-I) or the pharmaceutically acceptable salt thereof is administered orally or via a nasogastric tube.

39. The method of any one of claims 32-38, wherein the compound of formula (F-I) is administered to the patient.

40. The method of any one of claims 32-39, further comprising administering to the patient one or more additional drugs or treatments for treating COVID-19.
